(19) United States
(12) Patent Application Publication
(10) Pub. No.: US 2019/0175511 A1
(43) Pub. Date: Jun. 13, 2019

(54) A Multi-Class Anti-Retroviral Composition

(71) Applicant: HETERO LABS LIMITED, Hyderabad, Telangana (IN)

(72) Inventors: Parthasarathi Reddy BANDI, Hyderabad, Telangana (IN); Khadgapathi PODILE, Hyderabad, Telangana (IN); Sunil Deviprasad TIWARI, Hyderabad, Telangana (IN); Ramarao NELLURI, Hyderabad, Telangana (IN); Atluri VAMSI KIRAN, Hyderabad, Telangana (IN)

(21) Appl. No.: 16/324,232

(22) PCT Filed: Jul. 28, 2017
(86) PCT No.: PCT/IB2017/054592

§ 371 Date: Feb. 8, 2019

(30) Foreign Application Priority Data
Aug. 8, 2016 (IN) IN201641026996

(51) Int. Cl. A61K 9/20; A61P 31/18; A61K 31/5377; A61K 31/34; A61K 9/28; A61K 31/5365

(52) U.S. Cl. CPC A61K 9/2095; A61P 31/18; A61K 31/5377; A61K 31/5365; A61K 9/2086; A61K 9/28; A61K 31/34

(57) ABSTRACT

The present invention is related to an anti-retroviral composition. In particular, the present invention relates to a solid oral composition comprising combination of multi-class drugs particularly darunavir, dolutegravir and cobicistat and process for preparing the same.

### PRIORITY

This patent application claims priority to Indian patent application number IN 201641026996, filed on Aug. 8, 2016, the contents of which are incorporated by reference herein in their entirety.

### FIELD OF THE INVENTION

The present invention relates to an anti-retroviral composition. In particular, the present invention relates to a tablet composition comprising combination of multi-class drugs particularly darunavir, dolutegravir and cobicistat and process for preparing the same.

### BACKGROUND OF THE INVENTION

HIV continues to be a major global public health issue. In the early years, HIV was unknown, feared, untreatable and often fatal. However, over the past three decades we have come a long way in our understanding of HIV, where it came from, how it evolved, and most importantly, how to treat and prevent it.

HIV attacks and destroys the infection-fighting CD4 cells of the immune system. Loss of CD4 cells makes it hard for the body to fight off infections. HIV medicines prevent HIV from multiplying (making copies of itself), which reduces the amount of HIV in the body. Having less HIV in the body gives the immune system a chance to recover.

Currently available anti-retroviral drugs include Nucleoside Reverse Transcriptase Inhibitors (NRTIs), Non-nucleoside Reverse Transcriptase Inhibitors (NNRTIs), Protease Inhibitors (PIs), Integrase inhibitors or integrase strand transfer inhibitors (INSTIs), Fusion Inhibitors, Entry Inhibitors—CCR5 co-receptor antagonist, HIV integrase strand transfer inhibitors and the like thereof.

Antiretroviral therapy (ART) is the use of HIV medicines to treat HIV infection. ART involves taking a combination of HIV medicines (called an HIV regimen) every day. ART is recommended for everyone with HIV. ART can't cure HIV, but it helps people with HIV live longer, healthier lives.

Combinations of antiretrovirals create multiple obstacles to HIV replication to keep the number of offspring low and reduce the possibility of a superior mutation. If a mutation that conveys resistance to one of the drugs being taken arises, the other drugs continue to suppress reproduction of that mutation. Combination therapies greatly increase the ease with which they can be taken, which in turn increases the consistency with which medication is taken and thus their effectiveness over the long-term. Because of the complexity of selecting and following a regimen and the potential for side effects there lies an importance of taking medications regularly to prevent viral resistance.

Commonly used NRTI drugs have numerous toxicities partly due to the fact that they are analogs of naturally occurring nucleotides and interfere with the activity of numerous cellular functions. Most current Highly Active Anti-retroviral therapy (HAART) regimens include combination of three or more anti-retroviral agents, in common consists of three drugs namely, 2 NRTIs a PI/NNRTI/INSTI (e.g: combination of 2 NRTIs (Abacavir+Lamivudine) and 1 INSTI (dolutegravir) is an approved dosage form).

The need to keep plasma levels above a minimum level and the pharmacokinetic properties of the HIV drugs, a frequent administration of relatively high doses is needed. The number of HIV drugs and the amount used in the dosage form pose a common problem referred to as ‘pill burden’. A high pill burden is undesirable resulting in patient incompliance due to which most of the patients do not take the entire dose and thus fail to comply with the prescribed dosage regimen. The problems associated with high pill burden are multiplied when a combination of various anti-retroviral agents are administered in combination with booster anti-retrovirals (e.g: cobicistat) to improve pharmacokinetics.

One limitation of most of the NRTI-sparing regimens has been the above discussed higher pill burden than more other standard regimens. However, the approvals of dolutegravir and the co-formulated darunavir/cobicistat, both with well-established antiviral activities, may allow for an effective NRTI-sparing regimen. A shift to darunavir, dolutegravir and cobicistat combination in virologically suppressed HIV-infected individuals has the potential to avoid NRTI-associated toxicity while maintaining virologic suppression.

Still there exists a need for the novel combinations of anti-retroviral drugs. Accordingly, inventors of the present invention had developed compositions comprising a protease inhibitor (darunavir), an integrase inhibitor (dolutegravir) and a CYPA3 inhibitor (cobicistat) that has a relatively small size contributing to the convenience of intake and thus help to overcome the problem associated high pill burden and also the multi-class combinations aid to combat the problems associated viral resistance.

Darunavir ethanolate is a protease inhibitor, described chemically as [(1S,2R)-3-[[(4-aminophenyl)sulfonyl](2-methylpropyl)amino]-2-hydroxy-1(phenylmethyl)propyl]-carbamic acid (3R,3aS,6aR)-hexahydrofuro[2,3-b]furan-3-yl ester monoethanolate.

Darunavir ethanolate is commercially available in US as 75 mg, 150 mg, 600 mg & 800 mg equivalent to base tablets and as equivalent to 100 mg base/ml oral suspension under the brand name PREZISTA®.

Cobicistat is a CYP3A inhibitor, described chemically as 1,3-thiazol-5-ylmethyl [(2R,5R)-5-{[(2S)-2[(methyl{[2-(propan-2-yl)-1,3-thiazol-4-yl]methyl}carbamoyl)amino]-4-(morpholin-4 yl)butanoyl]amino}-1,6-diphenylhexan-2-yl].

Cobicistat is commercially available in US as 150 mg tablets under the brand name TYBOST®.

Combination of darunavir ethanolate and cobicistat is commercially available in the US market as 800 mg equivalent base/150 mg under the brand name PREZCOBIX®.

U.S. Pat. No. 5,843,946 & 7,700,645 discloses darunavir and its solvates.

PCT Publication No. 2009/013356 assigned to Tibotec disclose tablet compositions comprising darunavir.

U.S. Pat. No. 8,148,374 assigned to Gilead discloses cobicistat substance.

PCT Publication No. 2009/135179 assigned to Gilead discloses composition comprising cobicistat and process for preparing the same.

PCT Publication No. 2013/004818 assigned to Janssen discloses compositions comprising darunavir and cobicistat.

Dolutegravir sodium is an integrase inhibitor, described chemically as (4R,12aS)-9-{[(2,4-difluorophenyl) methyl]carbamoyl}-4-methyl-6,8-dioxo-3,4,6,8,12,12a-hexahydro-2H-pyrido[1′,2′:4,5]pyrazino [2,1-b][1,3]oxazin-7-olate sodium.

Dolutegravir sodium is commercially available in US as 10 mg, 25 mg & 50 mg equivalent to base tablets under the brand name TIVICAY®.

U.S. Pat. No. 8,129,385 assigned to VIIV Healthcare discloses dolutegravir substance.

### SUMMARY OF THE INVENTION

The present invention relates to pharmaceutical anti-retroviral composition. In particular, the present invention relates to solid oral compositions, comprising combination of multi-class drugs particularly, darunavir, dolutegravir and cobicistat and process of preparing the same.

One embodiment of the present invention is related to a pharmaceutical composition comprising darunavir, dolutegravir and cobicistat with one or more pharmaceutically acceptable excipients.

Another embodiment of the present invention is related to a pharmaceutical tablet composition comprising a) an intragranular portion comprising combination of darunavir and dolutegravir and one or more pharmaceutically acceptable excipients and b) an extragranular portion comprising of cobicistat and one or more pharmaceutically acceptable excipients.

An another embodiment of the present invention is related to pharmaceutical unitary tablet composition comprising a) 800 mg-1200 mg of darunavir or its pharmaceutically acceptable salt thereof, b) 50 mg of dolutegravir or its pharmaceutically acceptable salt thereof, c) 150 mg of cobicistat and d) one or more pharmaceutically acceptable excipients, wherein the total tablet weight ranges from 1600 mg to 1750 mg.

One another embodiment of the present invention is related to a bilayer tablet composition comprising darunavir, dolutegravir, cobicistat and one or more pharmaceutically acceptable excipients.

Yet another embodiment of the present invention is related to process of preparing a pharmaceutical tablet comprising the following steps (a) granules of darunavir were prepared by wet granulation (b) granules of dolutegravir were prepared by wet granulation (c) granules of step (a) and step (b) were mixed for a specified period of time (d) mixture of granules obtained in step (c) were mixed with extragranular cobicistat and one or more pharmaceutically acceptable excipients (e) blend of step (d) was compressed into tablets and (e) finally, tablets obtained in step (d) were film coated.

Further embodiment of the present invention is related to a pharmaceutical composition used in the treatment of HIV infection in a patient in need thereof.

### DETAILED DESCRIPTION OF THE INVENTION

The present invention relates to solid oral composition comprising combination of multi-class products particularly, darunavir, dolutegravir and cobicistat and process of manufacturing the same.

The term “active agent” as used herein according to the present invention refers to “darunavir”, “dolutegravir” and “cobicistat”.

The term “darunavir” as used herein according to the present invention includes darunavir in the form of free base or a pharmaceutically acceptable salt, solvate or ester thereof. Preferably, darunavir ethanolate.

The term “dolutegravir” as used herein according to the present invention includes dolutegravir in the form of free base or a pharmaceutically acceptable salt or solvate thereof.

Preferably, dolutegravir sodium.

The term “cobicistat” as used herein according to the present invention includes cobicistat in the form of free base or a pharmaceutically acceptable salt thereof. Preferably, cobicistat base.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

The term “excipient” means a pharmacologically inactive component such as a diluent, a binder, a disintegrant, a glidant, a lubricant, etc of a pharmaceutical product. The excipients that are useful in preparing a pharmaceutical composition are generally safe, non-toxic and are acceptable for veterinary as well as human pharmaceutical use. Reference to an excipient includes both one and more than one such excipient.

The term “composition” or “pharmaceutical composition” or “solid oral composition” or “dosage form” as used herein synonymously include solid dosage forms such as tablets, capsules, granules, pellets and the like meant for oral administration.

Pharmaceutical compositions according to the present invention are in the form of monolithic or bi-layer tablets.

The term “monolithic” as used anywhere in the present invention means a single layered tablet composition comprising of darunavir, dolutegravir, cobicistat and one or more pharmaceutically acceptable excipients.

The term “multi-class” as used anywhere in the present invention means anti-retroviral drugs belonging to various categories described in terms of pathway or mechanism of action of the drug. Preferably, multi-class compounds according to the present invention belong to protease inhibitors (e.g: Darunavir), integrase inhibitors (e.g: Dolutegravir) and CYPA3 inhibitors (e.g; Cobicistat).

One embodiment of the present invention is related to a pharmaceutical composition comprising darunavir, dolutegravir and cobicistat with one or more pharmaceutically acceptable excipients.

One another embodiment of the present invention comprises a multi-class combination of drugs comprising: a) a protease inhibitor consisting of darunavir or its pharmaceutically acceptable salt thereof, b) an integrase inhibitor consisting of dolutegravir or its pharmaceutically acceptable salt thereof, c) a CYPA3 inhibitor cobicistat and d) one or more pharmaceutically acceptable excipients.

In one aspect, the present invention is related to a pharmaceutical tablet composition comprising a) an intragranular portion comprising combination of darunavir and dolutegravir and one or more pharmaceutically acceptable excipients and b) an extragranular portion comprising of cobicistat and one or more pharmaceutically acceptable excipients.

Alternatively, present invention is related to a tablet composition comprising a) an intragranular portion comprising a mixture of granules comprising darunavir and granules comprising dolutegravir b) an extragranular portion comprising of cobicistat and one or more pharmaceutically acceptable excipients.

Compositions according to the present invention are prepared either by direct compression or granulation techniques namely wet granulation and dry granulation. Preferably, wet granulation.

Suitable granulating medium to prepare granules by wet granulation according to the present invention include binder solution or suspension comprising binder and a solvent. Alternatively, granulation can be carried by using solvent alone.

Solvents include but are not limited to purified water, tertiary-butyl alcohol, isopropyl alcohol, dichloromethane, methanol, methylene chloride and the like and combinations thereof.

According to one aspect, tablets of the present invention are manufactured by a process comprising steps of:
a) Mannitol, sodium starch glycolate and povidone were sifted through mesh #30,
b) dolutegravir sodium and materials of step (a) were sifted through mesh #30,
c) darunavir ethanolate and materials of step (b) were sifted through mesh #30,
d) blend of step (c) was granulated using purified water to get the desired granules,
e) obtained granules in step (d) were dried at 60° C. temperature and sifted through mesh #20,
f) silicon dioxide adsorbed cobicistat was sifted through mesh #40,
g) silicified microcrystalline cellulose, colloidal silicon dioxide and crospovidone together were sifted through mesh #40,
h) materials of step (f) and (g) were blended together for 10 mins,
i) sodium stearyl fumarate was sifted through mesh #40,
j) blend of step (h) was lubricated with sifted sodium stearyl fumarate of step (i),
k) lubricated blend of step (j) was compressed into tablets,
l) finally, tablets obtained in step (k) were film coated.

In another aspect, the pharmaceutical tablet compositions of the present invention were prepared by a process comprising steps of:

### Part 1—Darunavir Granulation:

a) Darunavir was sifted through a mesh #40,
b) granulating medium was prepared by dissolving hypromellose in purified water,
c) sifted darunavir of step (a) was granulated using the medium prepared in step (b)
d) granules obtained in step (c) were dried at 50° C. temperature,
e) dried granules of step (d) were sifted through mesh#30 to obtain desired size granules.

### Part 2—Dolutegravir Granulation:

a) Dolutegravir, mannitol and sodium starch glycolate were sifted through mesh#30,
b) microcrystalline cellulose and povidone were sifted through mesh #40
c) materials of step (a) and (b) were sifted together through mesh#30 and mixed for 10 mins,
d) blend of step (c) was granulated using purified water
e) granules obtained in step (d) were dried at 60° C. temperature,
f) dried granules of step (e) were sifted through mesh #35 to obtain desired size granules.

### Part 3—Extra-Granular Sifting:

a) silicon dioxide adsorbed cobicistat was sifted through mesh #40
b) silicified microcrystalline cellulose, colloidal silicon dioxide and crospovidone were co-sifted through mesh #40,
c) materials of step (a) and (b) were blended together for 10 mins,
d) dry mix the granules obtained in Part 1 and Part 2 for 10 mins,
e) pre-lubricate the mixed granules of step (d) with the blend of step (c)
f) sodium stearyl fumarate was sifted through mesh #40
g) pre-lubricated blend of step (e) was lubricated finally with sifted sodium stearyl fumarate of step (f),
h) compress the lubricated blend of step (g) into tablets, and
i) finally, tablets obtained in step (h) were film coated.

One another embodiment of the present invention is related to pharmaceutical unitary tablet composition comprising a) 800 mg-1200 mg of darunavir or its pharmaceutically acceptable salt thereof, b) 50 mg of dolutegravir or its pharmaceutically acceptable salt thereof, c) 150 mg of cobicistat and d) one or more pharmaceutically acceptable excipients, wherein the total tablet weight ranges from 1600 mg to 1750 mg.

The pharmaceutical tablet composition according the present invention comprise excipients selected from diluents, binders, disintegrants, lubricants, glidants and combinations thereof.

Diluents include but are not limited to starches, modified starches, lactose, dibasic calcium phosphate, tribasic calcium phosphate, microcrystalline cellulose, silicified microcrystalline cellulose, mannitol, calcium carbonate, calcium sulfate, talc, sugar, magnesium carbonate, magnesium oxide and the like or combinations thereof.

Binders include but are not limited to hydroxypropyl cellulose, low-substituted hydroxypropyl cellulose, hydroxypropyl methylcellulose or hypromellose, polyvinyl pyrrolidone (povidone), pregelatinized starch, powdered acacia, gelatin, guar gum, carbomers and the like or combinations thereof.

Disintegrants include but are not limited to croscarmellose sodium, sodium starch glycolate, crospovidone, polacrillin potassium, microcrystalline cellulose, polyvinylpyrrolidone, carboxymethyl cellulose calcium, starches such as corn starch, potato starch and modified starches, clays, bentonite, microcrystalline cellulose and the like or combinations thereof.

Lubricants include but are not limited to talc, magnesium stearate, calcium stearate, zinc stearate, stearic acid, palmitic acid, sodium stearyl fumarate, carnauba wax, hydrogenated vegetable oils, mineral oil, polyethylene glycols, and the like or combinations thereof.

Glidants include but are not limited to colloidal silicon dioxide, other forms of silicon dioxide, such as aggregated silicates, hydrated silica, magnesium silicate, magnesium trisilicate, talc, and the like or combinations thereof.

Advantageously, the tablet compositions of the present invention comprising three active ingredients particularly darunavir, dolutegravir and cobicistat, still can be formulated in an acceptable size that account for a reduced total tablet weight ranging from 1600 mg to 1750 mg. The said size and weight of the tablet dosage form thus can overcome the effect of pill burden.

One other embodiment of the present invention relates to a pharmaceutical composition comprising darunavir, dolutegravir, cobicistat and one or more excipients, wherein composition is in the form of tablet having a length of 20 to 22 mm, width of 8 to 11 mm and thickness of 7.0 to 8.5 mm.

One another embodiment of the present invention is related to a bilayer tablet composition, wherein
a) first layer comprises darunavir pre-lubricated with cobicistat and one or more pharmaceutically acceptable excipients and
b) second layer comprises granules of dolutegravir and one or more pharmaceutically acceptable excipients.

The solid oral dosage forms of the present invention are film coated with an aqueous or non aqueous solution comprising film forming polymers and one or more of plasticizers, opacifiers, anti-tacking agents, coloring agents and the like and combinations thereof.

A film coat on the tablet provides an elegant appearance, protects from moisture and further contributes to the ease with which it can be swallowed.

Film coating composition according to the present invention is polymer based. Suitable polymers include alkyl celluloses such as methyl cellulose, ethyl cellulose, hydroxypropyl cellulose, hydroxypropyl methyl cellulose, cellulose acetate, poly vinyl alcohol, polyvinyl acetate, poly vinyl chloride, polyvinyl pyrrolidone and the like and combinations thereof.

Plasticizers include but are not limited to glyceryl monostearate, triethyl citrate, macrogols, lactic acid, lactic acid acetamide, sorbitol, glycerin, triacetin, acetyl triethyl citrate, acetyl tributyl citrate, polyvinylpyrrolidone, triethylene glycol, ethylene glycol monooleate, acetylated monoglycerides, cetyl alcohol and other hydrogenated oils and waxes, as well as polyethylene glycol and the like or combinations thereof.

Yet another embodiment of the present invention is related to process of preparing a pharmaceutical tablet comprising the step of (a) granules of darunavir were prepared by wet granulation (b) granules of dolutegravir were prepared by wet granulation (c) granules of step (a) and step (b) were mixed for a specified period of time (d) mixture of granules obtained in step (c) were mixed with extragranular cobicistat and one or more pharmaceutically acceptable excipients (e) blend of step (d) was compressed into tablets and (e) finally, tablets obtained in step (d) were film coated.

Further embodiment of the present invention is related to a pharmaceutical composition used in the treatment of HIV infection in a patient in need thereof.

Certain specific aspects and embodiments of this invention are described in further detail by the examples below, which are provided only for the purpose of illustration and are not intended to limit the scope of the invention in any manner.

### EXAMPLES

### Example 1

### Single Layer Tablet Composition:

| | |
|---|---|
| Ingredient | mg/tab |

Intragranular Portion
Darunavir Granulation

| | |
|---|---|
| Darunavir ethanolate | 867.28 |
| Hypromellose | 13.20 |
| Purified water | q.s |

Dolutegravir Granulation

| | |
|---|---|
| Dolutegravir sodium | 52.60 |
| Microcrystalline cellulose | 59.00 |
| Mannitol | 140.40 |
| Sodium starch glycolate | 15.00 |
| Povidone | 15.00 |
| Purified water | q.s |

Extragranular Portion

| | |
|---|---|
| Cobicistat on silicon dioxide | 288.00 |
| Silicified Microcrystalline cellulose | 164.52 |
| Colloidal silicon dioxide | 17.00 |
| Crospovidone | 51.00 |
| Sodium stearyl fumarate | 17.00 |
| Core tablet weight | 1700.00 |

Film coating with Opadry

| | |
|---|---|
| Coated tablet weight | 1742.50 |

### Brief Manufacturing Process:

### Part 1—Darunavir Granulation:

a) Darunavir was sifted through a mesh #40,
b) granulation medium was prepared by dissolving hypromellose in purified water,
c) sifted darunavir of step (a) was granulated using the medium prepared in step (b)
d) granules obtained in step (c) were dried at 50° C. temperature,
e) dried granules of step (d) were sifted through mesh#30 to obtain desired size granules.

### Part 2—Dolutegravir Granulation:

a) Dolutegravir, mannitol and sodium starch glycolate were sifted through mesh#30,
b) silicified microcrystalline cellulose and povidone were sifted through mesh #40
c) materials of step (a) and (b) were sifted together through mesh#30 and mixed for 10 mins,
d) blend of step (c) was granulated using purified water
e) granules obtained in step (d) were dried at 60° C. temperature,
f) dried granules of step (e) were sifted through mesh #35 to obtain desired size granules.

### Part 3—Extra-Granular Sifting:

a) silicon dioxide adsorbed cobicistat was sifted through mesh #40
b) silicified microcrystalline cellulose, colloidal silicon dioxide and crospovidone were co-sifted through mesh #40,
c) materials of step (a) and (b) were blended together for 10 mins,
d) dry mix the granules obtained in Part 1 and Part 2 for 10 mins,
e) pre-lubricate the mixed granules of step (d) with the blend of step (c)
f) sodium stearyl fumarate was sifted through mesh #40
g) pre-lubricated blend of step (e) was lubricated finally with sifted sodium stearyl fumarate of step (f),
h) compress the lubricated blend of step (g) into tablets, and
i) finally, tablets obtained in step (h) were film coated.

### Comparative Dissolution Data:

### Dissolution Test Conditions:

Apparatus: USP apparatus II (Paddle type)
rpm: 100 rpm
pH: 3.0
Media: 0.05M sodium phosphate buffer containing 2% Tween 20
Volume:: 900 ml

TABLE 1
Comparative dissolution profile:

| | % drug released | | | | | |
|---|---|---|---|---|---|---|
| | | | | Innovator plain marketed tablets | | |
| Time in | Example 1 (Single layer tablet) | | | Darunavir | Cobicistat | Dolutegravir |
| minutes | Darunavir | Cobicistat | Dolutegravir | (Prezista ®) | (Tybost ®) | (Tivicay ®) |
| 10 | 51 | 91 | 83 | 41 | 86 | 76 |
| 15 | 64 | 93 | 90 | 56 | 87 | 84 |
| 20 | 71 | 94 | 94 | 63 | 87 | 88 |
| 30 | 81 | 95 | 96 | 74 | 88 | 90 |
| 45 | 88 | 95 | 98 | 82 | 89 | 93 |
| Infinity | 92 | 96 | 101 | 86 | 91 | 96 |

### Example 2

### Single Layer Tablet Composition:

| | |
|---|---|
| Ingredient | mg/tab |

Intra-granular Portion

| | |
|---|---|
| Darunavir ethanolate | 867.28 |
| Dolutegravir sodium | 52.60 |
| Mannitol | 54.98 |
| Sodium starch glycolate | 32.00 |
| Povidone (K-30) | 42.50 |
| Purified water | q.s |

Extragranular Portion

| | |
|---|---|
| Cobicistat on silicon dioxide | 288.00 |
| Silicified microcrystalline cellulose | 177.64 |
| Colloidal silicon dioxide | 17.00 |
| Crospovidone | 51.00 |
| Sodium stearyl fumarate | 17.00 |
| Core tablet weight | 1600.00 |

Film coating with Opadry

| | |
|---|---|
| Coated tablet weight | 1642.50 |

### Brief Manufacturing Process:

a) Mannitol, sodium starch glycolate and povidone were sifted through mesh #30,
b) dolutegravir sodium and materials of step (a) were sifted through mesh #30,
c) darunavir ethanolate and materials of step (b) were sifted through mesh #30,
d) blend of step (c) was granulated using purified water to get the desired granules,
e) obtained granules in step (d) were dried at 60° C. temperature and sifted through mesh #20,
f) silicon dioxide adsorbed cobicistat was sifted through mesh #40,
g) silicified microcrystalline cellulose, colloidal silicon dioxide and crospovidone together were sifted through mesh #40,
h) materials of step (f) and (g) were blended together for 10 mins,
i) sodium stearyl fumarate was sifted through mesh #40,
j) blend of step (h) was lubricated with sifted sodium stearyl fumarate of step (i),
k) lubricated blend of step (j) was compressed into tablets,
l) finally, tablets obtained in step (k) were film coated.

### Example 3

### Bi-Layer Tablet Composition:

| | |
|---|---|
| Ingredient | mg/unit |

Layer-I

| | |
|---|---|
| Darunavir ethanolate | 867.28 |
| Silicified microcrystalline cellulose | 354.693 |
| Crospovidone | 25.00 |

Prelubrication

| | |
|---|---|
| Cobicistat on colloidal silicon dioxide | 288.00 |
| Colloidal silicon dioxide | 16.667 |

Lubrication

| | |
|---|---|
| Magnesium stearate | 4.360 |

Layer-II

| | |
|---|---|
| Dolutegravir sodium | 52.60 |
| Microcrystalline cellulose | 63.00 |
| Mannitol | 140.40 |
| Sodium starch glycolate | 15.00 |
| Povidone (K-30) | 15.00 |

Granulation

| | |
|---|---|
| Purified water | q.s |

Prelubrication

| | |
|---|---|
| Sodium starch glycolate | 6.00 |

Lubrication

| | |
|---|---|
| Sodium stearyl fumarate | 8.00 |
| Total core tablet weight | 1856.00 |

Coating with Opadry

| | |
|---|---|
| Coated tablet weight | 1902.00 |

### Brief Manufacturing Process:

### Preparation of Layer 1—(Darunavir+Cobicistat):

a) Darunavir ethanolate, silicified microcrystalline cellulose and crospovidone were sifted through mesh #40
b) colloidal silicon dioxide loaded cobicistat and Colloidal silicon dioxide were co-sifted through mesh #40
c) materials of step (a) were pre-lubricated with materials of step (b)
d) magnesium stearate was sifted through mesh #40
e) pre-lubricated material of step (c) was lubricated finally with sifted magnesium stearate of step (d)

### Preparation of Layer 2—(Dolutegravir Granules):

a) Dolutegravir, mannitol and sodium starch glycolate were sifted through mesh#30,
b) microcrystalline cellulose and povidone were sifted through mesh #40
c) materials of step (a) and (b) were sifted together through mesh#30 and mixed for 10 mins,
d) blend of step (c) was granulated using purified water
e) granules obtained in step (d) were dried at 60° C. temperature,
f) dried granules of step (e) were sifted through mesh #35 to obtain desired size granules.

### Bilayer Tablet Compression:

Lubricated blend of Layer 1 and Layer 2 were compressed to obtain bilayer tablets and finally the tablets were film coated.

1. A pharmaceutical composition comprising darunavir, dolutegravir, cobicistat, and one or more pharmaceutically acceptable excipients.

2. The pharmaceutical composition of claim 1 in the form of a tablet, the tablet comprising
a) an intragranular portion comprising the darunavir, the dolutegravir, and one or more intragranular pharmaceutically acceptable excipients, and
b) an extragranular portion comprising the cobicistat and one or more extragranular pharmaceutically acceptable excipients.

3. A pharmaceutical unitary tablet composition comprising:
a) 800 mg-1200 mg of darunavir or a pharmaceutically acceptable salt thereof,
b) 50 mg of dolutegravir or a pharmaceutically acceptable salt thereof,
c) 150 mg of cobicistat, and
d) one or more pharmaceutically acceptable excipients,
wherein the total tablet weight ranges from 1600 mg to 1750 mg.

4. The pharmaceutical composition of claim 1, wherein one or more excipients are selected from diluents, binders, disintegrants, lubricants, glidants, and combinations thereof.

5. The pharmaceutical composition of claim 1, wherein the darunavir is darunavir ethanolate and the dolutegravir is dolutegravir sodium.

6. A pharmaceutical composition comprising darunavir, dolutegravir, cobicistat and one or more pharmaceutically acceptable excipients, wherein composition is in the form of tablet having a length of 20 to 22 mm and thickness of 7.0 to 8.0 mm.

7. The pharmaceutical composition of claim 1, wherein the composition is in the form of tablet having a total weight ranging from 1600 mg to 1750 mg.

8. The pharmaceutical composition of claim 3, comprising:
Ingredient
Intragranular Portion
Darunavir Granulation
Darunavir Ethanolate
Hypromellose
Purified water
Dolutegravir Granulation
Dolutegravir Sodium
MCC
Mannitol
Sodium starch glycolate
Povidone
Purified water
Extragranular Portion
Cobicistat on silicon dioxide
Silicified MCC
Colloidal silicon dioxide
Crospovidone
Sodium stearyl fumarate

9. A process of preparing a pharmaceutical tablet composition, comprising:
a) sifting darunavir through a mesh,
b) preparing a granulation medium by dissolving hypromellose in purified water,
c) granulating the sifted darunavir of step (a) in the medium prepared in step (b) and drying and sifting the granules to obtain uniform size darunavir granules,
d) separately sifting dolutegravir and one or more intragranular excipients,
e) granulating the materials of step (d) using purified water and drying and sifting the granules to obtain uniform size dolutegravir granules,
f) mixing the darunavir granules of step (c) and the dolutegravir granules of step (e),
g) sifting and blending extragranular cobicistat and one or more pharmaceutically acceptable extragranular excipients,
h) mixing the mixture of granules obtained in step (f) and the blend of step (g),
i) compressing the blend of step (h) into tablets,
j) film coating the finally, tablets obtained in step (i).

10. A method of treating HIV infection in a patient in need thereof comprising administering the pharmaceutical composition according to claim 1.

11. A method of treating HIV infection in a patient in need thereof comprising administering the pharmaceutical composition according to claim 3.

12. The pharmaceutical composition of claim 3, wherein one or more excipients are selected from diluents, binders, disintegrants, lubricants, glidants, and combinations thereof.

13. The pharmaceutical composition of claim 6, wherein the composition is in the form of a tablet having a total weight ranging from 1600 mg to 1750 mg.
